(19) United States
(12) Patent Application Publication
(10) Pub. No.: US 2010/0178365 A1
(43) Pub. Date: Jul. 15, 2010

(54) EPICATECHIN DEFICIENT GREEN TEA

(76) Inventor: Dae-Kyu SONG, Daegu (KR)

Correspondence Address: JHK LAW, P.O. BOX 1078, LA CANADA, CA, 91012-1078, US

(21) Appl. No.: 12/691,834

(22) Filed: Jan. 22, 2010

Related U.S. Application Data
(63) Continuation of application No. PCT/IB2008/003389, filed on Jul. 23, 2008.
(60) Provisional application No. 60/951,387, filed on Jul. 23, 2007.
(60) Provisional application No. 60/951,394, filed on Jul. 23, 2007.

(30) Foreign Application Priority Data
Sep. 11, 2007 (KR) 10-2007-0092193

(51) Int. Cl. A61K 36/82; A61P 9/10; A61P 31/00; A61P 29/00; A61P 35/00

(52) U.S. Cl. 424/729

(57) ABSTRACT

The present application describes a method of eliminating gallated catechins, especially epigallocatechin gallate (EGCG), epicatechin gallate (ECG), as well as parathion, from green tea extraction by using at least one out of ethyl acetate, Cetyl Trimethyl Ammonium Bromide (CTAB), cation exchange resin, anion exchange resin, C18 bead, PEG bead and mPEG. And also, this method can be applied in the method of adding at least one of pre-mentioned components, when green tea is directly ingested in the form of an extract solution, leaf or powder. This is to be useful for people with metabolic disorders such as diabetes and obesity because of the resolved toxicity. This method is to maximize the beneficial luminal effect and to minimize the harmful circulatory effect of gallated catechins.

### CROSS-REFERENCE TO RELATED APPLICATIONS

The present application is a continuation of PCT Application No. PCT/IB2008/003389, filed Jul. 23, 2008, which claims the benefit of priority to U.S. Provisional Application Ser. Nos. 60/951,387, filed Jul. 23, 2007 and 60/951,394, filed Jul. 23, 2007, and Korean Patent Application No. 10-2007-0092193, filed Sep. 11, 2007, which are incorporated by reference herein in their entirety.

### BACKGROUND OF THE INVENTION

1. Field of the Invention

The present invention relates to modified green tea extract. The present invention also relates to green tea extract lacking epicatechins such as (−)-epigallocatechin-3-gallate (EGCG) or (−)-epicatechin-3-gallate (ECG) and also parathion. The present invention also relates to methods of making green tea extract that is lacking catechins to prevent unexpected modulation of KATP channels and heart cell excitation. The present invention relates to methods of eliminating harmful substances such as gallated catechin, including epigallocatechin gallate (EGCG) and epicatechin gallate (ECG), and parathion which is an agricultural chemical, from green tea extracts and of producing purified drinks and powders by removing the components that have been revealed to worsen the conditions of diabetes and obesity.

2. General Background and State of the Art

Camellia sinensis and its extracted substances, catechins are known to have many beneficial effects such as anti-cancerous, anti-atherosclerotic, anti-lipogenic, anti-inflammatory and anti-pathogenic effects. Camellia sinensis contains epicatechins, in particular EGCG, EGC (epigallocatechin), ECG and EC (epicatechin). EGCG is the most dominant component having both anti-oxidant or oxidative effects in cells, controlling enzyme activation, interfering with promoter actions and membrane binding proteins of nucleic acids and ion channels. However, there have been few reports about EGCG having any toxic potential. And also the concentration of EGCG was 10-100 μM in data saying that EGCG are beneficial. To reach serum EGCG level to 10 μM, we have to drink 15 cups of green tea in the fasting state.

Most of the protein kinase activation is controlled by ATP competitive or ATP noncompetitive actions with flavonoids such as catechins, indicating that side effects, at least in a long-term manner, could usually happen in the body.

The ATP binding site that inhibits ATP-sensitive K (K_{ATP}) channels is specific to these protein kinases. Recent studies show that ATP α-, β- and γ-phosphate tails interact with the inward-rectifying potassium channel R201, K185 and R50 residues, respectively. ATP adenine tail N6 atom mostly interacts with E179 and R301.

It is known that K_{ATP} channels have their physiological role through a linkage between membrane excitability and their target function. It controls the secretion of insulin from the β islet cells of the pancreas, and thereby contributes to the maintenance of serum glucose level.

The K_{ATP} channel is an octamer composed of four inwardly-rectifying potassium (Kir) subunits and four sulfonylurea receptors (SUR). The K_{ATP} channel is inhibited by the ATP that acts on the Kir6.2 subunit and is activated by MgADP via SUR. Phosphatidylinositol polyphosphates (PIP), such as PIP2 and PIP3, and long chain acyl-CoA play a major role in activating K_{ATP} channel via the Kir6.2 subunit. It is known that the sensitivities of ATP and PIP at Kir6.2 are increased when the SUR subunit is present.

The inventive purpose is to remove substances such as gallated catechins that are diabetic toxins and parathion which is an agricultural chemical from edible plant extracts such as green tea. Also, another purpose of the invention is to create a safer functional green tea drink or powder by providing a removal method and making it safer to drink green tea.

### SUMMARY OF THE INVENTION

The present invention is directed to a plant extract, in particular, a tea leaf extract composition substantially lacking or substantially free of epicatechin or parathion. By “substantially free” of epicatechin or parathion, it is meant that the epicatechin or parathion substance is present in 0.0% to 2.0% percent by weight, or 0.0 to 1.5%, or 0.0 to 1.2%, or 0.0 to 1.1%, or 0.0 to 1.0%, or 0.0 to 0.9%, or 0.0 to 0.8%, or 0.0 to 0.7%, or 0.0 to 0.6%, or 0.0 to 0.5%, or 0.0 to 0.4%, or 0.0 to 0.3%, or 0.0 to 0.2%, or 0.0 to 0.1% of the extract. In another aspect, the invention is directed to a plant extract, in particular, tea leaf extract comprising 50.0 to 70.0 percent by weight epicatechin or parathion or 30.0 to 50.00 percent by weight epicatechin or parathion, or 0.0 to 30.0 percent by weight epicatechin or parathion. Preferably, the epicatechin to be removed may be, without limitation, (−)-epigallocatechin-3-gallate (EGCG) or (−)-epicatechin-3-gallate (ECG). The tea may be, without limitation, green tea or oolong tea or any other tea that contains epicatechin or parathion.

In another aspect, the invention is directed to a method of using a tea extract that contains 0.0 to 2.0 percent by weight of epicatechin for preventing excitation of cardiac or beta cells, which excitation would otherwise be caused by administration of a composition such as tea that contains catechins.

In another aspect, the invention is directed to a method of using a tea leaf that contains less than 70 percent by weight of epicatechin for preventing excitation of cardiac or beta cells, which excitation would otherwise be caused by administration of a composition such as tea that contains catechins.

Further, in another aspect, the epicatechin-free composition may include any number of other ingredients, so long as the composition is substantially free of epicatechin or parathion. Such additional ingredients may include compounds that help in treating ischemic heart disease and/or diabetes, such as taurine.

In another aspect, the invention is directed to a tea bag containing a substantially epicatechin or parathion free tea, in particular substantially epicatechin or parathion free green tea.

1. In one aspect, the invention is directed to a method of reducing concentration of epicatechins, in particular EGCG or ECG, or parathion from green tea or green tea extracts.

2. In another aspect, the invention in directed to decreasing harmful effect of EGCG or EGC or parathion when green tea or drinks containing green tea components are ingested by removing EGCG, ECG or parathion from the green tea or green tea extracts.

3. In another aspect, the invention is directed to apparatus and chemicals used for reducing EGCG, ECG or parathion content from green tea.

4. In yet another aspect, the invention is directed to a method for preventing symptoms of ischemic heart disease, obesity and diabetes that is associated with ingesting green tea or green tea product that comprises EGCG or ECG, comprising ingesting a green tea product in which EGCG or ECG is removed.

5. In yet another aspect, the invention is directed to modulating KATP channel activity in a subject comprising contacting the KATP channel with EGCG or ECG.

In one aspect, the present invention is directed to a plant extract composition processed so as to be substantially free of epicatechin or parathion. The plant is edible and may be a tea leaf, in particular, green tea leaf. The amount of epicatechin or parathion present in the extract may be in an amount of 0.0 to 2.0% by weight. The plant extract may be housed in a tea bag. A beverage drink may include the plant extract composition.

In another aspect, the invention is directed to a method of removing toxins from a plant extract comprising contacting the plant extract with a substance which binds to or dissolves the toxin, and separating out the substance from the plant extract, thereby leaving a plant extract which is substantially free of the toxin. The toxin may be catechin or parathion. The catechin may be gallated catechin, which may include EGCG or ECG. In one aspect, the substance that is used to eliminate the toxins from the plant extract may be ethyl acetate, Cetyl Trimethyl Ammonium Bromide (CTAB), cation exchange resin, anion exchange resin, C18 bead, PEG bead or mPEG.

In one aspect, the ethyl acetate may be contacted with green tea extract to form a mixture at a ratio of 50-200 ethyl acetate to 100 green tea extract (vol/vol), and then the used ethyl acetate may be removed from the mixture.

In another aspect, the CTAB may be contacted with green tea extract to form a mixture at a ratio of 0.05 to 0.1 CTAB to 100 green tea extract (wt/wt), and then the used CTAB may be removed from the mixture.

In another aspect, the cation exchange resin may be contacted with green tea extract to form a mixture at a ratio of 0.05-100 cation exchange resin to 100 green tea extract (wt/wt), and then the used cation exchange resin may be removed from the mixture. In particular, the cation exchange resin may be activated by 4-6M NaCl solution, 1-2.5M Ca(O₂CCH₃) solution or 2-4M KCl solution.

In another aspect, the C18 bead may be contacted with green tea extract to form a mixture at a ratio of 1-50 C18 bead to 100 green tea extract (wt/wt), and then the used C18 bead may be removed from the mixture.

In another aspect, the PEG bead may be contacted with green tea extract to form a mixture at a ratio of 0.01-50 PEG bead to 100 green tea extract (wt/wt), and then the used PEG bead may be removed from the mixture.

In another aspect, the mPEG may be contacted with green tea extract to form a mixture at a ratio of 0.01-200 mPEG to 100 green tea extract (wt/wt).

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows change of blood glucose concentration of rats during intraperitoneal glucose tolerance test (2 g/kg). Each epicatechin was injected intravenously 30 min before the glucose loading. High blood glucose level was exclusively seen in the EGCG and ECG injected group.

FIG. 2 is the result of blood glucose level measured during oral glucose tolerance test after a normal adult male was given medication of green tea extraction (350 ml, ECG content about 680 mg) 1 hour before oral glucose (75 g/150 ml). PEG group was given the same green tea extraction, but together with 0.42 mM or 2.3 mM of PEG.

FIG. 3 shows glucose uptake ability at cultured adipocytes, myocytes and hepatocytes. We can see that basal glucose uptake ability and insulin stimulated glucose uptake ability decreased based on the given EGCG concentration.

FIG. 4 shows blood glucose level in rats as time passes after intravenous injection of 100 mg green tea extraction liquid lyophilization powder in saline per kg (containing about 10 mg EGCG) in green tea group and in PEG pretreated group the same green tea extraction liquid lyophilized powder which was pretreated with PEG for elimination of gallated catechins was injected and after 30 minutes glucose (2 g/kg) was injected into the peritoneum. Similar blood glucose level was seen between PEG pretreated group and the control group.

FIG. 5 depicts the effect of the green tea extracts on blood glucose level during oral glucose tolerance test in rats. Green tea extracts and/or PEG was orally administered immediately before the glucose loading (2 g/kg). Note that green tea group and green tea plus PEG group showed less blood glucose levels compared to control and only PEG-ingested groups.

FIG. 6 is a HPLC graph showing the detected parathion in 0.2 g/10 ml green tea.

FIG. 7 is a HPLC graph showing the quantity change of parathion in green tea extract after treatment with ethyl acetate.

FIG. 8 is another graph showing the quantity change of parathion in green tea extract after treatment with cation exchange resin.

FIG. 9 is a HPLC graph showing the quantity change of parathion in green tea extract after treatment with C18 bead.

### DETAILED DESCRIPTION OF THE PREFERRED EMBODIMENTS

In the present application, “a” and “an” are used to refer to both single and a plurality of objects.

EGCG and ECG with gallate ester groups inhibited K_{ATP} channels via allosteric interactions with phospholipids of the lipid membrane. But the effective EGCG concentration was more than 10 μM. Further, EGCG is the only green tea epicatechin that decreases the sensitivity of ATP and PIP to the K_{ATP} channel. By this mechanism, we concluded that EGCG caused decrease in insulin secretion in response to glucose in the β-cell of pancreas. Patients with diabetes or people with a risk factor for diabetes should be careful because the effective concentration of EGCG for this mechanism could be 1-10 μM, which is an achievable concentration when a person drinks 2-8 cups of green tea while fasting.

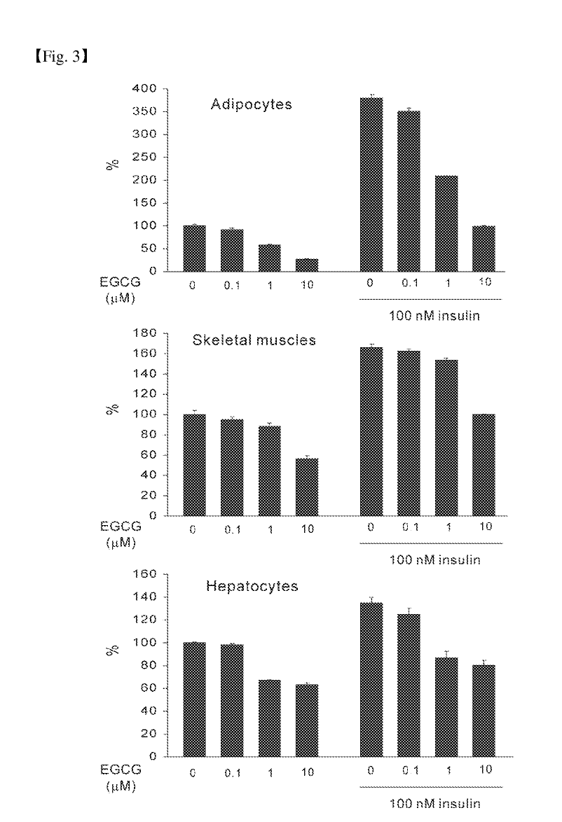

Gallated catechins EGCG and ECG inhibited normal glucose uptake mechanism after a meal into hepatocytes, adipocytes and muscle cells which play an important role in blood glucose clearance (FIG. 3). If the glucose clearance after a meal is not effective as stated above, long-term hyperglycemia will occur and will lead to emergence or worsening of diabetes and its complications such as vascular damage.

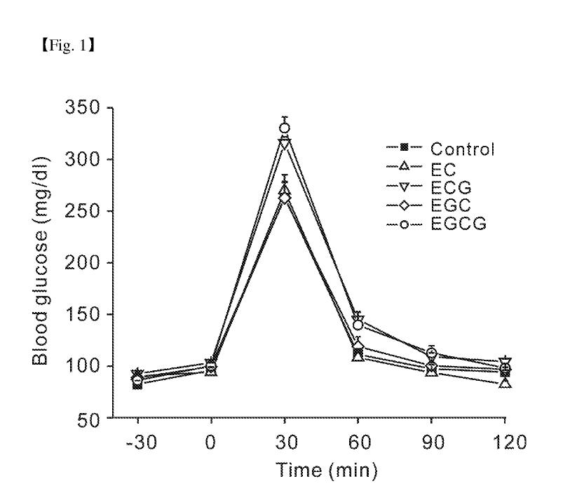

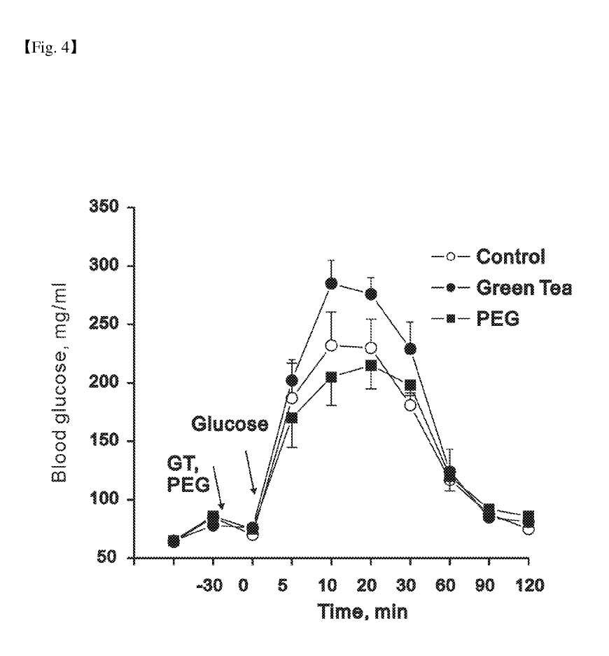

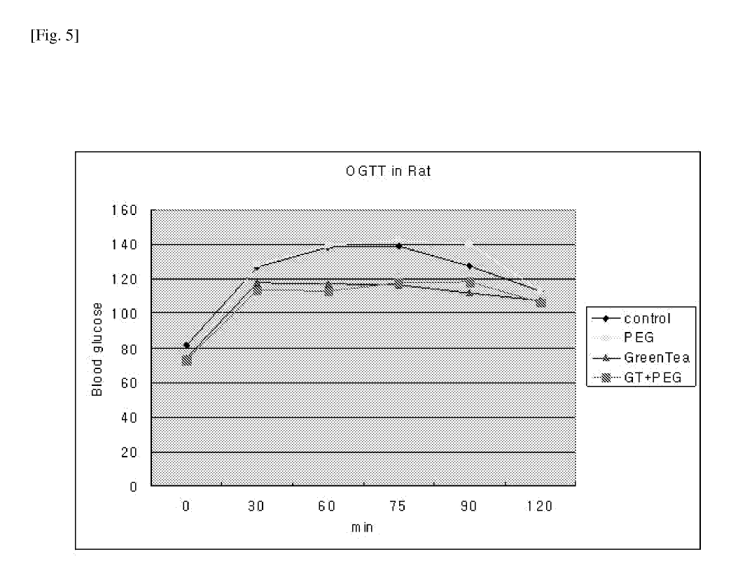

These phenomena can occur after commonly drinking green tea because it can occur when the blood EGCG level is lower than 10 μM. Unlike the action of the K_{ATP} channel, the glucose intolerance caused by green tea can happen by various types of gallated catechins, not only EGCG but also ECG (FIG. 1). Therefore, to prevent this harmful effect of gallated catechins in the circulation, elimination of gallated catechins from green tea extracts before ingestion (FIG. 4) or ingestion together with one of the elimination methods (FIG. 5) provides diminution of gallated catechins absorbed into the circulation. The latter concepts are further recommended by the fact that gallated catechins, which are harmful in the circulation via delaying blood glucose removal, can inhibit glucose and lipid absorption into the circulation from the alimentary tract (FIG. 5), which is beneficial mechanism against diabetes and obesity.

The method used in this invention to remove gallated catechins, which includes without limitation epigallocatechin gallate (EGCG), epicatechin gallate (ECG), and parathion involves a step of using ethyl acetate, Cetyl Trimethyl Ammonium Bromide (CTAB), cation exchange resin, anion exchange resin, Bondesil-C18 bead, PEG bead (Novasyn TG hydroxyl resin) and/or mPEG.

In detail, in one embodiment, EGCG, ECG and parathion-removing methods include the step of mixing green tea extraction with ethyl acetate at a ratio of 100 to 50-200 (volume to volume) and the step of removing ethyl acetate from the extract. The mixing step should be carried out for about 5 to 20 minutes.

The process of removing EGCG, ECG and parathion may be carried out by directly adding CTAB to the green tea extraction. In this situation, CTAB is added to the green tea extraction at a ratio of green tea extract 100 to CTAB 0.05-0.1 (weight to weight).

In another embodiment, the toxin removal process may be carried out by passing the extraction through a filter paper or tube containing cation exchange resins. The cation exchange resins may be added at a ratio of green tea extraction 100 to cation exchange resins 0.05-100 (weight to weight). The cation exchange resins may be activated by a 4 to 6M HCl solution or a 4 to 6M NaCl solution, 1 to 2.5M Ca(O₂CCH₃) solution or a 2 to 4M KCl solution.

The EGCG, ECG and parathion removing method may be carried out by directly adding the cation exchange resins to the green tea extraction. In this situation, green tea extraction may be mixed with cation exchange resins at a ratio of green tea extract 100 to cation exchange resin 0.05-100 (Wt/Wt). The used cation exchange resins are then removed from the green tea extraction. The mixing may be carried out for 5 to 30 minutes.

In another embodiment, the toxin removal process may be carried out by passing the extraction through a filter paper or tube containing anion exchange resins. The anion exchange resins may be added at a ratio of green tea extraction 100 to anion exchange resins 1-50 (weight to weight). The anion exchange resin may be activated by a 2 to 6M NaOH solution, 2 to 6 M KOH solution, 2 to 6M NaCl solution, or 2 to 4M KCl solution.

The EGCG, ECG and parathion removing method may be carried out by directly adding the anion exchange resins to the green tea extraction. In this situation, green tea extraction may be mixed with anion exchange resins at a ratio of green tea extract 100 to anion exchange resin 1-50 (weight to weight). The used anion exchange resins are then removed from the green tea extraction. The mixing may be carried out for 5 to 30 minutes.

In another embodiment, the removal process may be carried out by passing the extraction through a filter paper or tube containing C18 beads. The C18 beads may be added at a ratio of green tea extraction 100 to C18 beads 1-50 (weight to weight).

The EGCG, ECG and parathion removing method may be carried out by directly adding the C18 beads to the green tea extraction. In this situation, green tea extraction may be mixed with the C18 beads at a ratio of green tea extract 100 to C18 beads 1-50 (weight to weight). The used C18 beads are then removed from the green tea extraction. The mixing may be carried out for 5 to 30 minutes.

In another embodiment, the removal process may be carried out by passing the extraction through a filter paper or tube containing PEG beads. The PEG beads may be added at a ratio of green tea extraction 100 to PEG beads 0.01-50 (weight to weight).

The EGCG, ECG and parathion removing method may be carried out by directly adding the PEG beads to the green tea extraction. In this situation, green tea extraction may be mixed with the PEG beads at a ratio of green tea extract 100 to PEG beads 1-50 (weight to weight). The used PEG beads are then removed from the green tea extraction.

The EGCG, ECG and parathion removing method may be carried out by directly adding mPEG (methoxy PEG) to the green tea extraction. In this situation, green tea extraction may be mixed with the mPEG directly at a ratio of green tea extract 100 to mPEG 0.01-200 (weight to weight). The mixing may be carried out for 0.1 to 5 minutes.

Green Tea Beverages

The production of the green tea beverage according to primary characteristics, includes the steps of preparing green tea extractions from dried leaves by water, removing ethyl acetate from the hydrophilic extraction, and eliminating the removed ethyl acetate.

The inventive green tea beverage producing method in view of other aspects includes the steps of preparing the hydrophilic green tea extraction, passing the extraction through a filtering paper that includes or is layered with cation exchange resins, anion exchange resins, C18 beads or PEG beads or a tube that is layered with them.

In another aspect, the inventive green tea beverage producing method includes the steps of preparing green tea extractions by water from dried leaves, adding and mixing CTAB, cation exchange resins, anion exchange resins, C18 beads or PEG beads with the extraction, and eliminating the cation exchange resins, anion exchange resins, C18 beads and PEG beads.

In another aspect, the inventive green tea beverage producing method includes the steps of preparing extractions from dried green leaves by water and adding and mixing mPEG with the extraction.

In another aspect, the inventive green tea powder producing method includes the steps of preparing extractions from dried green tea leaves by water, adding and mixing cation exchange resins, anion exchange resins, C18 bead or PEG bead with the extraction, eliminating the cation exchange resins, anion exchange resins, C18 bead or the PEG bead, and lyophilizing the extracts, and turning them into powder forms. The powderizing step may be carried out using well-known methods.

In another aspect, the inventive green tea powder producing method includes the steps of adding mPEG to the powder (0.01-200:100 Wt/Wt). This mixing procedure is carried out for about 0.1 to 5 minutes.

Further Detailed Description of the Invention

Selective removal of ECG and EGCG from green tea leaves or green tea extracts may be accomplished by solid-liquid extraction, liquid-liquid extraction, liquid-liposome extraction or combination of those extractions. For extraction processes, some chemicals can be added to enhance the selectivity and extraction efficiency.

In solid-liquid extraction, organic solvents such as methanol, ethanol, propanol, isopropanol, diethyl ether, hexane, acetone, ethyl acetate, acetonitrile, dichloromethane and toluene may be added to the extracting water. Also, extracting temperature can be changed from 50° C. to 100° C. with variable extraction times.

In liquid-liquid extraction, water insoluble organic solvents such as diethyl ether, hexane, ethyl acetate, dichloromethane, and toluene may be used.

In liquid-liposome extraction, lipids which can form either micelle or liposome can be added to extracting water.

Selective removal of ECG and EGCG from green tea leaves or green tea extracts can be accomplished by hydrolysis of gallate ester. Hydrolysis can be carried out by either thermal decomposition or chemical decomposition. For the chemical hydrolysis processes, inorganic acids or bases may be used.

Selective removal of ECG, EGCG and parathion from green tea leaves or green tea extracts can be accomplished by the separation techniques such as absorption, excursion or partition chromatography.

In one aspect, the invention comprises formulations of green tea polyphenols, in particular, catechins, for the production of specialized tea products for hyperglycemia patients.

The green tea utilized was Seijak from Boseongnokchamyoungga (Chunnam, South Korea). HPLC grade water, acetonitrile, ethyl acetate, methanol, and ethanol were obtained from Samchun Chemical, South Korea. Analytical grade trifluoroacetic acid was obtained from Acros. Ion exchange resins were obtained from Aldrich (USA). Epicatechin, Epicatechin gallate, Epigallocatechin, and Epigallocatechin gallate were obtained from Sigma (USA).

The inventive purification methods to remove gallated catechins, including without limitation epigallocatechin gallate (EGCG), epicatechin gallate (ECG) and parathion from the green tea extraction include without limitation settling the extraction with ethyl acetate, with Cetyl Trimethyl Ammonium Bromide (CTAB), with cation exchange resins, with anion exchange resins, with C18 bead, with PEG bead and finally with mPEG.

The relative amounts of catechins were determined by High Pressure Liquid Chromatography (HPLC) using aqueous acetonitrile containing 0.1% trifluoroacetic acid as eluent. Quantification of catechins was performed using calibration curves obtained using pure catechins.

Eliminating Toxic Substances by Treating Green Tea Extraction with Ethyl Acetate (EA)

Ethyl acetate is liquid in form and to use it for EGCG, ECG and parathion elimination it needs to be mixed with the green tea extraction for a certain amount of time. The ethyl acetate is included with a ratio of the tea extraction 100 volume versus 50-200 volume of EA. There are no problems in the case of the quantity of EA exceeding 200, but the efficiency rate of removal does not increase as much. Preferably, ethyl acetate is mixed with the amount of 80-120 volume relative to the extraction 100 volume. The mixing time for the extraction and ethyl acetate may be about 2 to 20 minutes, during which time EGCG, ECG and parathion are selectively dissolved. Ethyl acetate may be separated from the extraction such as by centrifugation or layer separation methods. As a result, EGCG, ECG and parathion in the extraction can be selectively removed. Other catechin substances are also dissolved but EGCG is the most dominant.

This method of removing EGCG, ECG and parathion can be included in the process of green tea production thus allowing the emergence of a better health benefiting green tea beverage.

Therefore, the method above can be accomplished by primarily preparing the extraction from dried leaves by using hot water and successively mixing ethyl acetate to the extraction and then removing ethyl acetate after the appropriate amount of time has passed.

Eliminating Toxic Substances by Treating Green Tea Extraction with Cetyl Trimethyl Ammonium Bromide (CTAB)

Another method to remove EGCG, ECG and parathion from green tea is to treat the green tea extraction with the solid substance cetyl trimethyl ammonium bromide (CTAB) by directly adding it to the green tea extraction and then centrifuging the extraction after an adequate amount of time has passed. CTAB is included at a ratio of tea extraction 100 versus 0.05-0.1 CTAB (Wt/Wt).

Eliminating Toxic Substances by Treating Green Tea Extraction with Cation Exchange Resins

Another way to remove EGCG, ECG and parathion is to use a filter paper or a tube containing cation exchange resins and passing the extraction through thus allowing selective EGCG, ECG and parathion clearance.

Alternatively, the cation exchange resin may be added directly to the extraction and passed through the filter thus clearing the toxic substances from the extraction.

The cation exchange resin amount used in this method may include the tea extraction 100 versus cation exchange resin 0.05-100 (Wt/Wt).

The layering includes simply putting the cation resins on the filtering paper in which the extraction is vertically moved and filtered.

Another method of using the cation resins includes first adding the resins directly to the extraction, then after the right amount of time has passed, clearing the resins by filtering. This method, compared to the method using layers, relatively has more contact surface and time with the extraction and therefore can save the amount of resins used.

This clearing method may be used in massive production by small scaled beverage stores or by consumers. For example, in small scaled production, a functional green tea beverage may be made by primarily producing the extraction by filtering and then additionally using the inventive method.

For the cation exchange resins used in this method 4 to 6M of HCl solution is used for activation. 4˜6M NaCl solution, 1˜2.5M Ca(O₂CCH₃) solution or 2˜4M KCl solutions also can be used for activation.

Eliminating Toxic Substances by Treating Green Tea Extraction with Anion Exchange Resins

An additional method to eliminate EGCG, ECG and parathion is to use solid anion exchange resin layered filtering paper or anion exchange resin layered tube and passing the green tea extraction through it resulting in EGCG, ECG and parathion clearance from the green tea extract. The layering includes simply putting the anion exchange resins on the filtering paper and moving the extraction vertically and filtering.

2˜6M NaOH can be used to activate the anion exchange resins or one or more of 2˜6M KOH, 2˜6M NaCl or 2˜4 KCl solution can be used to activate the resins.

The anion exchange resin amount used may be in a ratio of resin 1-50 versus tea extraction 100 by weight.

Directly adding the anion exchange resin to the extraction and then filtering and thus clearing the tea extraction of the anion exchange resin is another method that can be used to clear the toxic substances. This method, compared to the method using layers, relatively has more contact surface and time with the extraction and therefore can save the amount of resins used. The anion exchange resin amount used may be in a ratio of resin 1-50 versus tea extraction 100 by weight.

This clearing method may be used in massive production by small scaled beverage stores or by consumers. For example, in small scaled production, a functional green tea beverage may be made by primarily producing the extraction by filtering and then additionally using the inventive method.

Eliminating Toxic Substances by Treating Green Tea Extraction with C18 Bead

Another method to selectively remove EGCG, ECG and parathion is to use a C18 bead layered filter paper or tube to pass the extraction. The layering includes simply putting the C18 beads on the filtering paper and moving the extraction vertically and filtering.

Another way to clear the toxic substance is to directly add the C18 bead to the extraction and clearing the C18 bead by filtering.

The amount of the C18 bead should be used at a ratio of C18 bead 1-50 versus the tea extraction of 100 (Wt/Wt).

Another method of using the C18 beads can be performed by first adding the beads directly to the extraction, then after the right amount of time has passed, clearing the beads by filtering. This method, compared to the method using layers, relatively has more contact surface and time with the extraction and therefore can save the amount of beads used.

This clearing method may be used in massive production by small scaled beverage stores or by consumers. For example, in small scaled production, a functional green tea beverage may be made by primarily producing the extraction by filtering and then additionally using the inventive method.

Eliminating Toxic Substances by Treating Green Tea Extraction with Polyethylene Glycol (PEG) Bead

Another method to selectively remove EGCG, ECG and parathion is to use a solid PEG bead layered filter paper or tube to pass the extraction.

The right amount of the PEG bead may be 0.01-50 weight versus the tea extraction of 100.

The layering includes simply putting the PEG bead on the filtering paper and moving the extraction vertically and filtering.

Another method of using the PEG beads can be performed by first adding the beads directly to the extraction, then after the right amount of time has passed, clearing the beads by filtering. This method, compared to the method of using layers, relatively has more contact surface and time with the extraction and therefore can save the amount of beads used.

This clearing method may be used in massive production by small scaled beverage stores or by consumers. For example, in small scaled production, a functional green tea beverage may be made by primarily producing the extraction by filtering and then additionally using the inventive method.

Eliminating Toxic Substances by Treating Green Tea or Green Tea Extraction with Methoxy Polyethylene Glycol (mPEG)

Another way to clear EGCG, ECG and parathion is to add and agitate the mPEG to the extraction and selectively clear the toxic materials.

The right amount of mPEG should be 0.01-200 weight versus the tea extraction of 100.

In this application, use of only cation exchange resins, anion exchange resins, C18 beads, PEG beads and mPEG are exemplified but also other resins, beads and other types of PEG macromolecules may be used by a person of skill in the art.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Removal of EGCG and ECG from Green Tea Extraction by Using Ethyl Acetate

We confirmed that EGCG and ECG were removable by separating ethyl acetate by centrifuging after mixing ethyl acetate with the green tea extract.

Quantitative analysis of the catechin content in the green tea extraction such as EGCG, ECG was done by HPLC (High Pressure Liquid Chromatography) using acetonitrile containing 0.1% trifluoroacetic acid as an expansion liquid.

Catechin quantity was obtained from the quantity curve with pure catechin at a known concentration.

Extraction was carried out in a 60° C. water for 5 minutes from dry green tea leaf (product name: sejac, manufacturer: bosung nok cha myoung ka, Jeonnam, Korea).

After removing solid leaf material with a 0.45 μm filter, 5 minutes of agitation was carried out in 60° C. water after adding 10 ml of ethyl acetate. Separation of the ethyl acetate layers was done by centrifugation and by gathering the water layer. Catechin content in the water layer was analyzed and the results are shown in Table 1.

TABLE 1
Relative ratio of catechin in green tea extract before and after
treatment with ethyl acetate

| | | | | | other | |
|---|---|---|---|---|---|---|
| | | | | | catechins | |
| | EGC (%) | EC (%) | EGCG (%) | ECG (%) | (%) | EGCG/EGC |
| untreated extracts | 32.3 ± 2.9 | 8.7 ± 0.6 | 37.5 ± 0.6 | 6.9 ± 0.8 | 14.6 ± 1.2 | 1.16 ± 0.16 |
| (average of 9 | | | | | | |
| separate | | | | | | |
| experiments) | | | | | | |
| extracts after | 54.3 ± 3.2 | 6.0 ± 0.9 | 17.9 ± 1.9 | 3.3 ± 1.9 | 18.4 ± 4.4 | 0.33 ± 0.11 |
| treatment with | | | | | | |
| ethyl acetate | | | | | | |
| (average of 4 | | | | | | |
| separate | | | | | | |
| experiments) | | | | | | |

We can see in Table 1 the content of EGCG and ECG were selectively decreased after the treatment with ethyl acetate.

### Comparative Example 1

### Eliminating EGCG and ECG by Using Ethyl Acetate Without Removing Solid Leaf Material

Extraction was carried out in 10 ml of 60° C. water for 5 minutes from the 0.2 g dry green tea leaf, and then without removing solid leaf material agitation was done at 10 ml 60° C. water after mixing it with 10 ml of ethyl acetate. We analyzed the catechin content and the results are shown on Table 2.

TABLE 2
Relative ratio of catechin in green tea extract before and after
treatment with ethyl acetate in the presence of green tea leaves

| | | | | | other | |
|---|---|---|---|---|---|---|
| | | | | | catechins | |
| | EGC (%) | EC (%) | EGCG (%) | ECG (%) | (%) | EGCG/EGC |
| untreated | 32.3 ± 2.9 | 8.7 ± 0.6 | 37.5 ± 0.6 | 6.9 ± 0.8 | 14.6 ± 1.2 | 1.16 ± 0.16 |
| extracts | | | | | | |
| (average of 9 | | | | | | |
| separate | | | | | | |
| experiments) | | | | | | |
| extracts after | 39.5 ± 1.9 | 6.3 ± 0.5 | 34.2 ± 1.8 | 3.3 ± 0.8 | 16.6 ± 0.9 | 0.87 ± 0.09 |
| treatment | | | | | | |
| with ethyl | | | | | | |
| acetate | | | | | | |
| (average of 5 | | | | | | |
| separate | | | | | | |
| experiments) | | | | | | |

As we can see in Table 2 with the presence of solid material there was almost no change in the EGCG content even after the ethyl acetate treatment. Without being bound by any theory to explain the results, one explanation for this is that the green tea leaf persistently provides EGCG. Therefore, to remove EGCG effectively green tea extraction should be prepared primarily after the elimination of solid material and then secondary process may proceed.

### Example 2

### Removal of EGCG and ECG Using CTAB

Extraction was done in 10 ml of 60° C. water for 5 minutes from the 0.2 g dry green tea leaf. And then filtering the leaf at a 0.45 fall filter agitation was done at a 60° C. for 5 minutes after adding CTAB. Centrifugation was done and precipitated CTAB. Catechin content was analyzed by High Pressure Liquid Chromatography (HPLC) using aqueous acetonitrile containing 0.1% trifluoroacetic acid as eluent. Quantifications of catechins were performed using calibration curves obtained from authentic catechins.

The amount of addition was 5 mg and 10 mg and the catechin content was analyzed in each case. The results are shown in Table 3.

TABLE 3
Relative ratio of catechin in green tea extraction before and after
treatment with CTAB

| | % EGC | % EC | % EGCG | % ECG | % Catechin | EGCG/ECG |
|---|---|---|---|---|---|---|
| Untreated extracts | 32.3 ± 2.9 | 8.7 ± 0.6 | 37.5 ± 2.3 | 6.9 ± 0.8 | 14.6 ± 1.2 | 1.16 ± 0.16 |
| (average of 9 | | | | | | |
| separate | | | | | | |
| experiments) | | | | | | |
| Extracts treated | 40.2 ± 1.4 | 11.1 ± 0.6 | 25.8 ± 2.0 | 3.2 ± 0.4 | 19.7 ± 0.6 | 0.64 ± 0.07 |
| with CTAB 5 mg | | | | | | |
| (average of 4 | | | | | | |
| separate | | | | | | |
| experiments) | | | | | | |
| Extracts treated | 44.2 ± 4.8 | 11.7 ± 1.7 | 19.4 ± 7.5 | 3.1 ± 2.1 | 21.6 ± 3.3 | 0.44 ± 0.23 |
| with CTAB 10 mg | | | | | | |
| (average of 4 | | | | | | |
| separate | | | | | | |
| experiments) | | | | | | |
| Extracts treated | 31.2 | 9.0 | 35.8 | 7.8 | 16.1 | 1.15 |
| with CTAB 20 mg | | | | | | |
| Extracts treated | 29.6 | 8.4 | 38.0 | 8.5 | 15.5 | 1.29 |
| with CTAB 50 mg | | | | | | |

### Example 3

### Removal of EGCG and ECG by Using Cation Exchange Resin

Extraction was done in 10 ml of 60° C. water for 5 minutes from the 0.2 g dry green tea leaf. Next, agitation at 60° C. for 5 minutes after adding cation exchange resin (Dowex 50Wx2-100) (activated in a 6M HCl solution) into the remaining extraction (filtered in a 0.45 fall filter) was carried out.

The relative amounts of catechins were determined by High Pressure Liquid Chromatography (HPLC) using aqueous acetonitrile containing 0.1% trifluoroacetic acid as eluent. Quantifications of catechins were performed using calibration curves obtained using pure catechins.

Cation exchange resin was used after it was activated in a pipe in which 6M HCl solution (20 times larger in volume than the resin's) was slowly flowing and cation exchange was washed with deionized water before its use.

After removing resin, catechin content in water soluble state was analyzed. The results are shown in Table 4.

TABLE 4
Relative ratio of the catechin in green tea extraction before and after
treatment with cation exchange resin

| | | | | | other | |
|---|---|---|---|---|---|---|
| | | | | | catechins | |
| | EGC (%) | EC (%) | EGCG (%) | ECG (%) | (%) | EGCG/EGC |
| untreated | 32.3 ± 2.9 | 8.7 ± 0.6 | 37.5 ± 0.6 | 6.9 ± 0.8 | 14.6 ± 1.2 | 1.16 ± 0.16 |
| extracts | | | | | | |
| (average of 9 | | | | | | |
| separate | | | | | | |
| experiments) | | | | | | |
| extracts after | 39.8 ± 1.4 | 10.7 ± 0.8 | 30.5 ± 1.8 | 3.6 ± 2.3 | 15.4 ± 1.5 | 0.80 ± 0.12 |
| treatment with | | | | | | |
| CER (0.5 g) | | | | | | |
| (average of 4 | | | | | | |
| separate | | | | | | |
| experiments) | | | | | | |
| extracts after | 40.4 | 9.8 | 29.8 | 5.2 | 14.8 | 0.70 |
| treatment with | | | | | | |
| CER (1.0 g) | | | | | | |
| (average of 2 | | | | | | |
| separate | | | | | | |
| experiments) | | | | | | |
| extracts after | 44.6 ± 7.7 | 11.3 ± 1.0 | 26.9 ± 3.0 | 3.9 ± 2.5 | 13.3 ± 2.9 | 0.60 ± 0.16 |
| treatment with | | | | | | |
| CER (2.0 g) | | | | | | |
| (average of 3 | | | | | | |
| separate | | | | | | |
| experiments) | | | | | | |

As we can see in Table 4, after using cation exchange resin (activated with 6M HCl) the decrease of EGCG and ECG is shown.

### Example 4

### Removal of EGCG and ECG by Using Cation Exchange Resin

Extraction was carried out in 10 ml of 60° C. water for 5 minutes from 0.2 g dry green tea leaf. Next, agitation was carried out at 60° C. for 5 minutes after adding 1.5 g cation exchange resin (activated in a 6M NaCl, 2.5M Ca(O₂CCH₃), 3.5M KCl solution) into the remaining extraction (filtered in a 0.45 fall filter).

The cation exchange resin was used after it was activated in a pipe in which 6M NaCl, 2.5 M Ca(O₂CCH₃) or 3.5 M KCl solution (20 times larger in volume than the resin's) was slowly flowing and cation exchange was washed with deionized water before its use.

After removing the resin, catechin content in a water soluble state was analyzed. The results are shown in Table 5.

TABLE 5
Relative ratio of catechin in green tea extraction before and after
treatment with cation exchange resin

| | | | | | other | |
|---|---|---|---|---|---|---|
| | EGC (%) | EC (%) | EGCG (%) | ECG (%) | catechins (%) | EGCG/EGG |
| untreated extracts | 32.3 ± 2.9 | 8.7 ± 0.6 | 37.5 ± 0.6 | 6.9 ± 0.8 | 14.6 ± 1.2 | 1.16 ± 0.16 |
| (average of 9 | | | | | | |
| separate | | | | | | |
| experiments) | | | | | | |
| extracts after | 39.0 | 9.2 | 17.3 | 1.3 | 33.2 | 0.44 |
| treatment with | | | | | | |
| cation activated | | | | | | |
| with 6M NaCl | | | | | | |
| (average of 2 | | | | | | |
| separate | | | | | | |
| experiments) | | | | | | |
| extracts after | 39.6 | 9.5 | 20.9 | 2.8 | 27.1 | 0.53 |
| treatment with | | | | | | |
| cation activated | | | | | | |
| with 2.5M | | | | | | |
| Ca(OAc)₂ | | | | | | |
| (average of 2 | | | | | | |
| separate | | | | | | |
| experiments) | | | | | | |
| extracts after | 39.4 | 9.5 | 16.7 | 1.6 | 32.8 | 0.43 |
| treatment with | | | | | | |
| cation activated | | | | | | |
| with 3.5M KCl | | | | | | |
| (average of 3 | | | | | | |
| separate | | | | | | |
| experiments) | | | | | | |

According to Table 5, the elimination effect of EGCG and ECG in Example 4 was more effective than in Example 3.

### Example 5

### Removal of EGCG and ECG with PEG Bead

Extraction was carried out from 0.2 g dried green tea leaf in 10 ml of 60° C. water for 5 minutes. It was then filtered by a 0.45 fall filter. And then 0.1 g of PEG bead was added.

Next, it was agitated at 60° C. for 5 minutes. After the agitation resin was removed by filter the catechin content in the water soluble fraction was measured using HPLC. The results are shown in Table 6.

TABLE 6
Relative ratio of catechin in green tea extraction before and after
treatment with PEG beads

| | | | | | other | |
|---|---|---|---|---|---|---|
| | | | | | catechins | |
| | EGC (%) | EC (%) | EGCG (%) | ECG (%) | (%) | EGCG/EGG |
| untreated | 32.3 ± 2.9 | 8.7 ± 0.6 | 37.5 ± 0.6 | 6.9 ± 0.8 | 14.6 ± 1.2 | 1.16 ± 0.16 |
| extracts | | | | | | |
| extracts after | 37 | 10 | 1 | <1 | 52 | 0.03 |
| treatment with | | | | | | |
| 0.1 g PEG bead | | | | | | |

### Example 6

### Removal of EGCG and ECG Using mPEG

Extraction from 30 g dried green tea leaf was done in 500 ml of 80° C. water for 3 minutes. After the green tea leaf was filtered in a 0.45 μm filter 7.6 g of mPEG was added. And then it was mixed for 2 minutes in ambient temperature. We presumed the absorption of EGCG indirectly by measuring the amount of glucose in blood during oral glucose challenge after a person drank this solution.

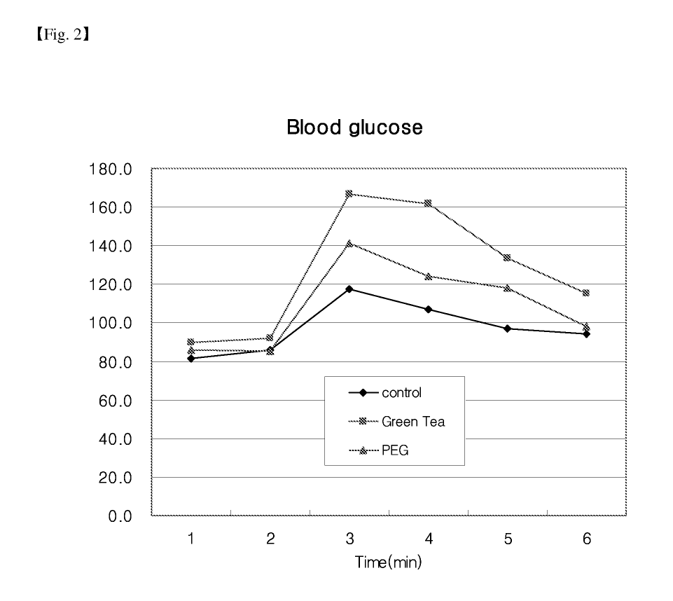

The fact that the increase in the amount of glucose in the blood by green tea is by EGCG and ECG was indicated by experimentation (FIG. 1). Accordingly, it is plausible that the reason of lower blood glucose in the group treated with green tea plus mPEG is due to the blocking effect of mPEG in absorption of EGCG and ECG from the alimentary tract (FIG. 2).

### Example 6

### Experiment to See Parathion Elimination Effect

1. Experimental Method

(1) Detection of Parathion in Green Tea Leaf

After 10 minutes of stirring of 0.2 g of dried green tea leaf in 10 ml of 100° C. water it was filtered and the green tea leaf was removed. Next the quantity of parathion was analyzed with HPLC

(2) Removing Parathion with Extraction Method

Standard solution was made by diluting the 17% parathion (commercially available) 100 times with methanol (manufactured by sam-jeon chemistry, Republic of Korea).

Solution diluted with methanol 100 times was analyzed with HPLC. Also the solution diluted by 100 times by adding 100 μl of standard solution into 9.9 ml of water was analyzed with HPLC.

20 minutes of agitation was done after adding 10 ml of ethyl acetate in the diluted solution. After separating it into ethyl acetate and water layer the parathion content was each measured with HPLC.

(3) Removing Parathion by Absorption Method

100 times diluted 17% parathion was used as a standard solution. Solution made by adding 10 μl of standard solution in 9.9 ml of water was analyzed with HPLC.

After adding 1 g of activated cation exchange resin into the previous solution, minutes of agitation was done. And then cation exchange resin was removed by filter. Next the amount of parathion remaining in the solution was measured.

After adding 2 g of activated cation exchange resin into the previous solution, 5 minutes of agitation was done. And then cation exchange resin was removed by filter. Next the amount of parathion remaining in the solution was measured by HPLC.

After adding 0.4 g of activated C18 bead into the previous solution, 5 minutes of agitation was done. And then C18 bead was removed by filtration. Next, the amount of parathion remaining in the solution was measured by HPLC.

2. Results

(1) Detection of Parathion Existing in Green Tea Leaf

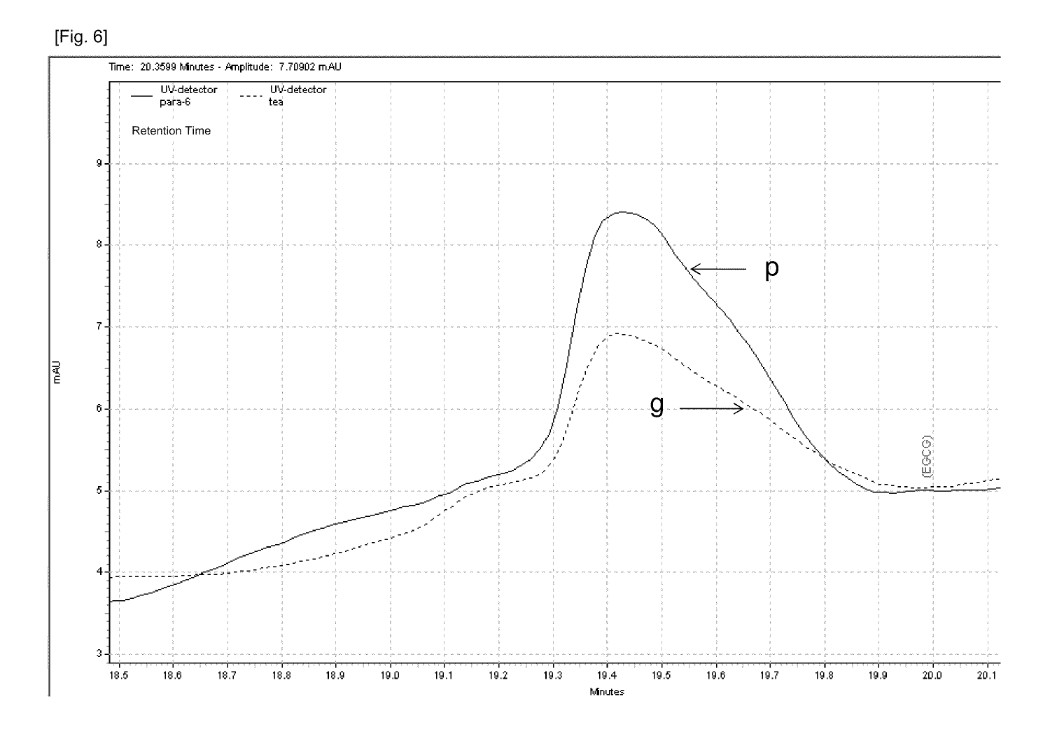

Status about extracted and detected parathion from 0.2 g/10 ml green tea is shown in FIG. 6.

FIG. 6 is a HPLC graph showing the amount of parathion remaining in 0.2 g/10 ml of green tea. A line indicated as “p” is for parathion 0.17 mg/L, and the line indicated as “g” is for green tea extracts. From the extractions from green tea a substance with the same retention time with parathion was detected.

Other analysis tools are needed to examine the exact structure of this material but still when we presumed it as a parathion the amount is lesser than 0.17 mg/L.

But again, considering 1 cup of green tea is 2 g green tea/100 ml water, the amount of parathion from a cup of green tea is almost the same as the daily intake limit (0.02 mg/kg/day) that FDA approved.

(2) Removing Parathion with Extraction Method

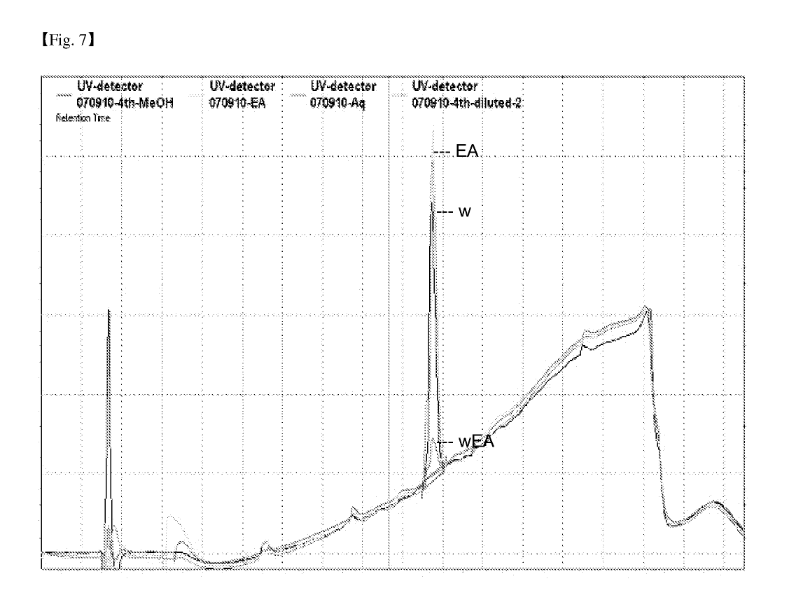

The result of eliminating parathion dissolved in the water is shown in FIG. 7.

FIG. 7 is a HPLC graph showing the quantity change of parathion by the method using ethyl acetate.

In FIG. 7, the line indicated as “w” refers to the water solution before it was treated with ethyl acetate, and the line indicate as “EA” refers to the ethyl acetate layer after the solution was treated with ethyl acetate. The line indicated as “wEA” refers to the water solution after it was treated with ethyl acetate.

(3) Removing Parathion with Absorption Method

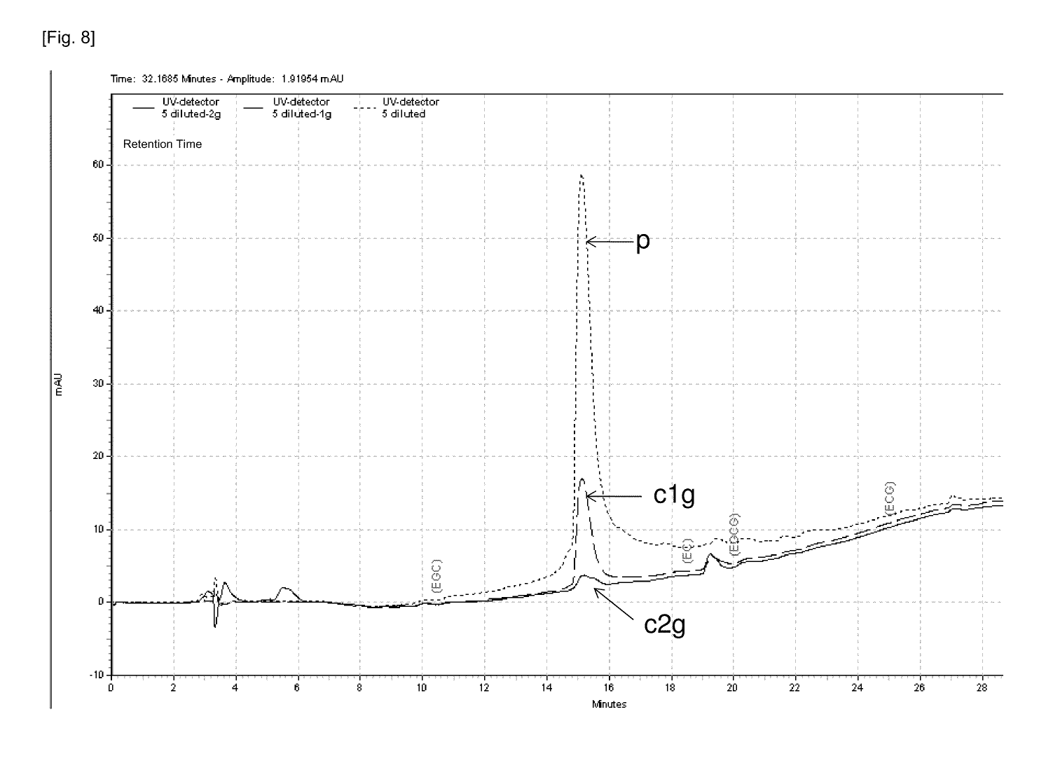

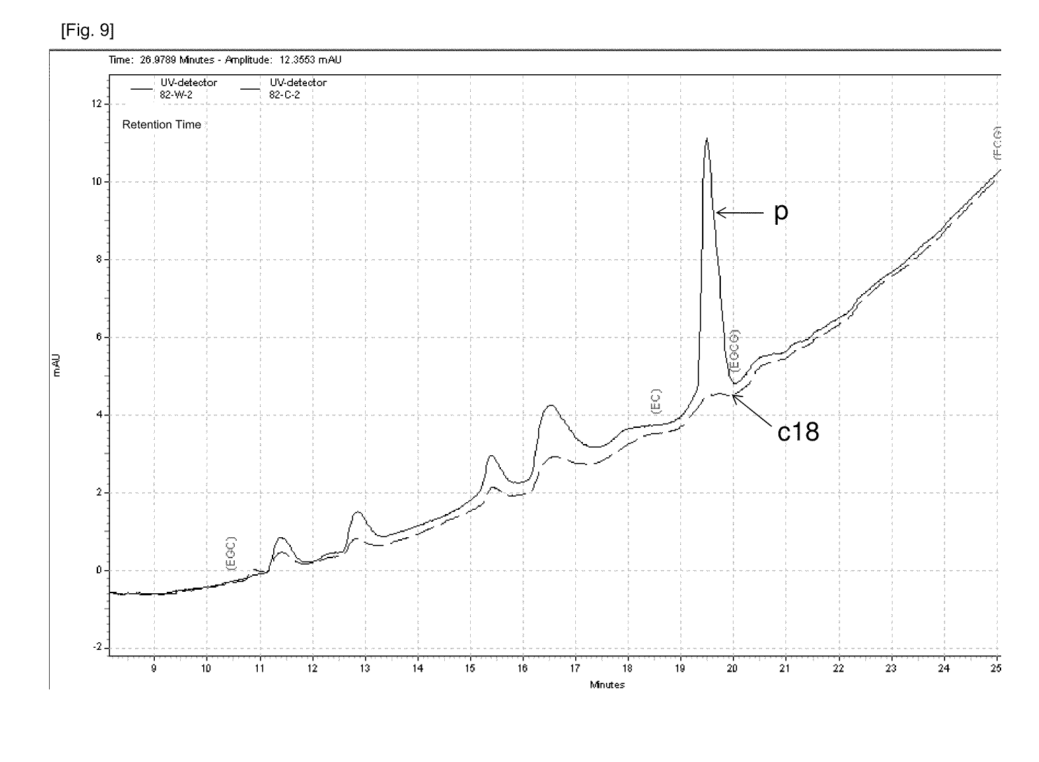

The result of removal of parathion dissolved in water with absorption methods are shown in FIGS. 8 and 9.

FIG. 8 is an HPLC graph showing the quantity change in parathion by treating with cation exchange resin.

The parathion amount in the solution decreased in proportion to the amount of ion exchange resin used. (parathion solution: line indicated as “p”, solution treated with 1 g of ion exchange resin: line indicated as “c1g”, Solution treated with 2 g of ion exchange resin: line indicated as “c2g”).

FIG. 9 is an HPLC graph showing the quantity change of parathion according to the use of C18 bead.

The amount of parathion in the solution was decreased by adding C18 bead. (parathion solution: line indicated as “p”, solution treated with 0.4 g of C18 bead: line indicated as “c18”).

According to this invention we can effectively and selectively remove EGCG, ECG and parathion from the green tea extraction liquid. So it is possible to develop an adequate green tea drink and green tea powder product to those who are vulnerable to diabetes with a help of this invention.

And also by removing parathion which is a harmful agricultural chemical it is possible to make a safer green tea drink from a green tea extract.

Finally applying this invention to the industry may provide healthful drink or food to the people.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

### REFERENCES

1. Qanungo, S., Das, M., Haldar, S., and Basu, A. (2005) Carcinogenesis 26, 958-967
2. Arts, I. C., Hollman, P. C., Feskens, E. J., Bueno de Mesquita, H. B., and Kromhout, D. (2001) Am J Clin Nutr 74, 227-232
3. Miura, Y., Chiba, T., Tomita, I., Koizumi, H., Miura, S., Umegaki, K., Hara, Y., Ikeda, M., and Tomita, T. (2001) J Nutr 131, 27-32
4. Kao, Y. H., Hiipakka, R. A., and Liao, S. (2000) Endocrinology 141, 980-987
5. Fiorini, R. N., Donovan, J. L., Rodwell, D., Evans, Z., Cheng, G., May, H. D., Milliken, C. E., Markowitz, J. S., Campbell, C., Haines, J. K., Schmidt, M. G., and Chavin, K. D. (2005) Liver Transpl 11, 298-308
6. Lu, H., Meng, X., and Yang, C. S. (2003) Drug Metab Dispos 31, 572-579
7. Ichikawa, D., Matsui, A., Imai, M., Sonoda, Y., and Kasahara, T. (2004) Biol Pharm Bull 27, 1353-1358
8. Lin, Y. L., and Lin, J. K. (1997) Mol Pharmacol 52, 465-472
9. Kawai, K., Tsuno, N. H., Kitayama, J., Okaji, Y., Yazawa, K., Asakage, M., Hori, N., Watanabe, T., Takahashi, K., and Nagawa, H. (2003) J Allergy Clin Immunol 112, 951-957
10. Hirasawa, M., and Takada, K. (2004) J Antimicrob Chemother 53, 225-229
11. Xia, J., Song, X., Bi, Z., Chu, W., and Wan, Y. (2005) Int J Mol Med 16, 943-950
12. Yun, S. Y., Kim, S. P., and Song, D. K. (2006) Eur J Pharmacol 541, 115-121
13. Hwang, J. T., Park, I. J., Shin, J. I., Lee, Y. K., Lee, S. K., Baik, H. W., Ha, J., and Park, O. J. (2005) Biochem Biophys Res Commun 338, 694-699
14. Garbisa, S., Sartor, L., Biggin, S., Salvato, B., Benelli, R., and Albini, A. (2001) Cancer 91, 822-832
15. Kuzuhara, T., Sei, Y., Yamaguchi, K., Suganuma, M., and Fujiki, H. (2006) J Biol Chem 281, 17446-17456
16. Baek, W. K., Jang, B. C., Lim, J. H., Kwon, T. K., Lee, H. Y., Cho, C. H., Kim, D. K., Shin, D. H., Park, J. G., Lim, J. G., Bae, J. H., Yoo, S. K., Park, W. K., and Song, D. K. (2005) Biochem Pharmacol 70, 1560-1567
17. Zhou, Q., Kwan, H. Y., Chan, H. C., Jiang, J. L., Tam, S. C., and Yao, X. (2003) Int J Mol Med 11, 261-266
18. Hagiwara, M., Inoue, S., Tanaka, T., Nunoki, K., Ito, M., and Hidaka, H. (1988) Biochem Pharmacol 37, 2987-2992
19. Dabrowski, M., Tarasov, A., and Ashcroft, F. M. (2004) J Physiol 557, 347-354
20. Trapp, S., Haider, S., Jones, P., Sansom, M. S., and Ashcroft, F. M. (2003) Embo J 22, 2903-2912
21. Antcliff, J. F., Haider, S., Proks, P., Sansom, M. S., and Ashcroft, F. M. (2005) Embo J 24, 229-239
22. Miki, T., Nagashima, K., Tashiro, F., Kotake, K., Yoshitomi, H., Tamamoto, A., Gonoi, T., Iwanaga, T., Miyazaki, J., and Seino, S. (1998) Proc Natl Acad Sci USA 95, 10402-10406
23. Wang, R., Liu, X., Hentges, S. T., Dunn-Meynell, A. A., Levin, B. E., Wang, W., and Routh, V. H. (2004) Diabetes 53, 1959-1965
24. Gumina, R. J., Pucar, D., Bast, P., Hodgson, D. M., Kurtz, C. E., Dzeja, P. P., Miki, T., Seino, S., and Terzic, A. (2003) Am J Physiol Heart Circ Physiol 284, H2106-2113
25. Ashcroft, F. M. (2005) J Clin Invest 115, 2047-2058
26. John, S. A., Weiss, J. N., and Ribalet, B. (2005) J Physiol 568, 931-940
27. Song, D. K., and Ashcroft, F. M. (2001) J Biol Chem 276, 7143-7149
28. Inagaki, N., Gonoi, T., Clement, J. P. t., Namba, N., Inazawa, J., Gonzalez, G., Aguilar-Bryan, L., Seino, S., and Bryan, J. (1995) Science 270, 1166-1170
29. Aguilar-Bryan, L., Nichols, C. G., Wechsler, S. W., Clement, J. P. t., Boyd, A. E., 3rd, Gonzalez, G., Herrera-Sosa, H., Nguy, K., Bryan, J., and Nelson, D. A. (1995) Science 268, 423-426
30. Inagaki, N., Gonoi, T., Clement, J. P., Wang, C. Z., Aguilar-Bryan, L., Bryan, J., and Seino, S. (1996) Neuron 16, 1011-1017
31. Isomoto, S., Kondo, C., Yamada, M., Matsumoto, S., Higashiguchi, O., Horio, Y., Matsuzawa, Y., and Kurachi, Y. (1996) J Biol Chem 271, 24321-24324
32. Han, J., Bae, J. H., Kim, S. Y., Lee, H. Y., Jang, B. C., Lee, I. K., Cho, C. H., Lim, J. G., Suh, S. I., Kwon, T. K., Park, J. W., Ryu, S. Y., Ho, W. K., Earm, Y. E., and Song, D. K. (2004) Am J Physiol Endocrinol Metab 287, E1008-1018
33. Han, J., Kim, N., Kim, E., Ho, W. K., and Earm, Y. E. (2001) J Biol Chem 276, 22140-22147
34. Shyng, S. L., and Nichols, C. G. (1998) Science 282, 1138-1141
35. Gribble, F. M., Tucker, S. J., Haug, T., and Ashcroft, F. M. (1998) Proc Natl Acad Sci USA 95, 7185-7190
36. Ribalet, B., John, S. A., Xie, L. H., and Weiss, J. N. (2006) J Physiol 571, 303-317
37. Lambert, J. D., and Yang, C. S. (2003) J Nutr 133, 3262S-3267S
38. Fan, Z., and Makielski, J. C. (1999) J Gen Physiol 114, 251-269
39. Kajiya, K., Kumazawa, S., and Nakayama, T. (2001) Biosci Biotechnol Biochem 65, 2638-2643
40. Mikhailov, M. V., Campbell, J. D., de Wet, H., Shimomura, K., Zadek, B., Collins, R. F., Sansom, M. S., Ford, R. C., and Ashcroft, F. M. (2005) Embo J 24, 4166-4175
41. Youdim, M. B. (2003) J Neural Transm Suppl, 73-88

What is claimed is:

1. An edible plant extract composition processed so as to be substantially free of epicatechin or parathion.

2. The plant extract composition according to claim 1, which is tea leaf.

3. The plant extract composition according to claim 2, which is green tea.

4. The plant extract composition according to claim 1, wherein the epicatechin or parathion is present in the extract in an amount of 0.0 to 2.0% by weight.

5. The plant extract composition according to claim 4, which is housed in a tea bag.

6. A beverage drink comprising the plant extract composition according to claim 1.

7. A method of removing toxins from a plant extract comprising contacting the plant extract with a substance which binds to or dissolves the toxin, and separating out the substance from the plant extract, thereby leaving a plant extract which is substantially free of the toxin.

8. The method according to claim 7, wherein the toxin is catechin or parathion.

9. The method according to claim 8, wherein the catechin is gallated catechin.

10. The method according to claim 9, wherein the gallated catechin is EGCG or ECG.

11. The method according to claim 7, wherein the plant extract is green tea extract.

12. The method according to claim 7, wherein, the substance is ethyl acetate, Cetyl Trimethyl Ammonium Bromide (CTAB), cation exchange resin, anion exchange resin, C18 bead, PEG bead or mPEG.

13. The method according to claim 12, wherein in the case of ethyl acetate, the ethyl acetate is contacted with green tea extract to form a mixture at a ratio of 50-200 ethyl acetate to 100 green tea extract (vol/vol), and then the used ethyl acetate is removed from the mixture.

14. The method according to claim 12, wherein in the case of CTAB, the CTAB is contacted with green tea extract to form a mixture at a ratio of 0.05 to 0.1 CTAB to 100 green tea extract (wt/wt), and then the used CTAB is removed from the mixture.

15. The method according to claim 12, wherein in the case of cation exchange resin, the cation exchange resin is contacted with green tea extract to form a mixture at a ratio of 0.05-100 cation exchange resin to 100 green tea extract (wt/wt), and then the used cation exchange resin is removed from the mixture.

16. The method according to claim 15, wherein the cation exchange resin is activated by 4-6M NaCl solution, 1-2.5M Ca(O₂CCH₃) solution or 2-4M KCl.

17. The method according to claim 12, wherein in the case of C18 bead, the C18 bead is contacted with green tea extract to form a mixture at a ratio of 1-50 C18 bead to 100 green tea extract (wt/wt), and then the used C18 bead is removed from the mixture.

18. The method according to claim 12, wherein in the case of PEG bead, the PEG bead is contacted with green tea extract to form a mixture at a ratio of 0.01-50 PEG bead to 100 green tea extract (wt/wt), and then the used PEG bead is removed from the mixture.

19. The method according to claim 12, wherein in the case of mPEG, the mPEG is contacted with green tea extract to form a mixture at a ratio of 0.01-200 mPEG to 100 green tea extract (wt/wt).

20. The method according to claim 19, wherein the ratio is 0.01-50 mPEG to 100 green tea extract (wt/wt).
